(19) United States
(12) Patent Application Publication
(10) Pub. No.: US 2018/0214686 A1
(43) Pub. Date: Aug. 2, 2018

(54) IMPLANTABLE MEDICAL DEVICE

(71) Applicant: CARDIAC PACEMAKERS, INC., St. Paul, MN (US)

(72) Inventors: Andrew L. De Kock, Ham Lake, MN (US); G. Shantanu Reddy, Minneapolis, MN (US); Christopher Alan Fuhs, Roseville, MN (US); Daniel J. Foster, Lino Lakes, MN (US); Peter Hall, Andover, MN (US); James K. Cawthra, JR., Ramsey, MN (US)

(73) Assignee: CARDIAC PACEMAKERS, INC., St. Paul, MN (US)

(21) Appl. No.: 15/882,130

(22) Filed: Jan. 29, 2018

Related U.S. Application Data
(60) Provisional application No. 62/452,537, filed on Jan. 31, 2017.

(51) Int. Cl. A61N 1/05; A61N 1/362; A61N 1/39; A61N 1/37

(52) U.S. Cl. CPC A61N 1/0504; A61N 1/37; A61N 1/3956; A61N 1/3621

(57) ABSTRACT

IMD devices and implantation methods are discussed and disclosed. Electrode structures may be employed to allow electrical stimulation to heart tissue and/or sense a physiological condition. Devices may be used for the placement of the electrode structures in a patient and facilitate the degree of contact between the electrode structures and the tissue of the patient.

### CROSS REFERENCE TO RELATED APPLICATIONS

The present application claims the benefit of and priority to U.S. Provisional Patent Application Ser. No. 62/452,537, filed on Jan. 31, 2017, titled IMPLANTABLE MEDICAL DEVICE, the disclosure of which incorporated herein by reference.

### BACKGROUND

The implantable defibrillator has been demonstrated to extend patient lives by treatment of potentially deadly arrhythmias. Over time, various efforts have been made to address complications associated with implantation of such devices. For example, early devices generally used epicardial patch electrodes implanted via thoracotomy, with attendant surgical morbidity and significant risks of failure of the epicardial patch electrodes and associated leads. The introduction of transvenous leads represented a major advance, avoiding the thoracotomy and improving reliability. However, lead failure remained a significant issue, as the lead attachment in the heart required the lead to flex with each heartbeat. The advent of subcutaneous defibrillators allows avoidance of these lead failure issues, with leads implanted beneath the skin and over the ribcage of the patient, and not subjected to repeated flexing with the beating heart.

However, subcutaneous defibrillators require higher energy for defibrillation, causing the pulse generators for such systems to be larger than their transvenous predecessors. Both bradycardia pacing and anti-tachycardia pacing are of limited utility as such pacing subcutaneously can be very uncomfortable for the patient. This has led to interest in further alternative designs and implant locations for implantable cardiac stimulus devices such as pacemakers and defibrillators. With such interest there is a need for new and alternative lead designs and anchoring devices.

### Overview

The present inventors have recognized, among other things, that certain new and alternative electrode structures may be employed to allow electrical stimulation to heart tissue and/or sense a physiological condition. Devices may be used for the placement of the electrode structures in a patient and may facilitate the degree of contact between the electrode structures and the tissue of the patient.

A first non-limiting example takes the form of a channel body for use in an implantable medical device, the channel body comprising a body having an outer surface and a longitudinal axis extending between a proximal end and a distal end, and a plurality of channels extending from the proximal end to the distal end of the body and radially spaced from one another, each of the channels being configured to receive an electrode structure therein such that the body electrically isolates the electrode structures from one another and enables contact between tissue of a patient and an electrode structure located in the lumen. Such an example may optionally comprise a lumen extending from the proximal end to the distal end along the longitudinal axis and configured to receive a positioning mechanism.

A second non-limiting example take the form of a lead assembly for use in an implantable medical device system comprising the channel body as in the first non-limiting example, a lead body extending from a proximal end to a distal end, the channel body secured at or near the distal end of the lead body, and a plurality of conductors extending through the lead body to the channel body which are coupled to the electrode structures.

Additionally or alternatively a third non-limiting example takes the form of a lead assembly as in the second non-limiting example wherein the lead assembly has a first length, and the channel body has a second length, and the second length is less than half of the first length.

Additionally or alternatively a fourth non-limiting example takes the form of a lead assembly as in the second non-limiting example wherein the lead assembly has a first length, and the channel body has a second length, and the second length is at least about 90% of the first length.

Additionally or alternatively a fifth non-limiting example takes the form of a lead assembly as in the second to fourth non-limiting examples further comprising an outer sheath having one or more portions made up of a conductive material, and one or more portions made up of a dielectric material.

Additionally or alternatively a sixth non-limiting example takes the form of a lead assembly as in the second to fifth non-limiting examples wherein the channels of the channel body extend in a longitudinal direction along the channel body.

Additionally or alternatively a seventh non-limiting example takes the form of a lead assembly as in the second to fifth non-limiting examples wherein the channels of the channel body extend helically about the channel body.

Additionally or alternatively an eighth non-limiting example takes the form of a lead assembly as in the second to seventh non-limiting examples comprising a connector at a proximal end thereof for coupling to an implantable medical device, the connector having contacts corresponding to one or more of the plurality of conductors.

A ninth non-limiting example takes the form of an implantable medical device comprising an implantable pulse generator housing operational circuitry for the implantable medical device system, and a lead assembly as in the eighth non-limiting example, wherein the implantable pulse generator comprises a port for receiving the connector of the lead assembly, the connector and port configured to electrically couple the plurality of conductors to the operational circuitry, and the operational circuitry is configured to deliver therapy using the electrode structures.

Additionally or alternatively a tenth non-limiting example takes the form of an implantable medical device as in the ninth non-limiting example wherein the implantable pulse generator is configured to deliver a pacing therapy by selecting a first electrode structure of the lead assembly for use in pacing therapy delivery, and selecting a second pacing electrode as an opposing electrode thereto, the implantable pulse generator is configured to deliver a defibrillation therapy by linking at least two of the plurality of electrode structures in common for use for defibrillation therapy delivery, and selecting a second defibrillation electrode as an opposing electrode thereto, further wherein the second pacing electrode is a second electrode structure of the lead assembly, an electrode coupled to a separate conductor from the plurality of conductors, or an electrode on the implantable pulse generator, and the second defibrillation electrode is one or more of, an electrode coupled to a separate conductor from the plurality of conductors, and/or an electrode on the implantable pulse generator.

Additionally or alternatively an eleventh non-limiting example takes the form of an implantable medical device as in the tenth non-limiting example wherein the operational circuitry is configured to measure impedances between one or more of the electrode structures of the lead assembly and determine from the measured impedances which of the electrode structures to use in pacing therapy delivery.

A twelfth non-limiting example takes the form of an implantable medical device comprising an implantable pulse generator housing operational circuitry for the implantable medical device system, and a lead assembly as in the eighth non-limiting example, wherein the implantable pulse generator comprises a port for receiving the connector of the lead assembly, the connector and port configured to electrically couple the plurality of conductors to the operational circuitry, and the operational circuitry is configured to sense cardiac activity using the electrode structures.

A thirteenth non-limiting example takes the form of an implantable retention mechanism (IRM) for use with an implantable lead, the IRM comprising a torsion tube extending from a proximal end to a distal end and configured to receive the implantable lead, and a securing structure, having at least one flap, located adjacent to the distal end of the torsion tube and configured to move from a pre-deployment state to a post-deployment state, wherein in the pre-deployment state the flap is compressed and in the post-deployment state the flap extends away from the torsion tube, wherein the securing structure has a proximal end coupled to the torsion tube and a distal end that is configured to push against a first portion of tissue of a patient causing an electrode structure disposed on the implantable lead to press against a second portion of tissue of the patient.

Additionally or alternatively a fourteenth non-limiting example takes the form of an IRM as in the thirteenth non-limiting example wherein the securing structure includes a plurality of flaps radially spaced around the torsion tube.

Additionally or alternatively a fifteenth non-limiting example takes the form of an IRM as in the thirteenth to fourteenth non-limiting examples wherein the at least one flap comprises a flap having a fan shape.

Additionally or alternatively a sixteenth non-limiting example takes the form of an IRM as in the thirteenth to fifteenth non-limiting examples wherein the at least one flap comprises a flap having a tine shape.

Additionally or alternatively a seventeenth non-limiting example takes the form of an IRM as in the thirteenth to sixteenth non-limiting examples further comprising a suture sleeve located adjacent to the proximal end of the torsion tube and including one or more channels to receive a suture for tying purposes to secure the IRM in a desired position in the patient with the flap deployed.

Additionally or alternatively an eighteenth non-limiting example takes the form of an IRM as in the seventeenth non-limiting example wherein the suture sleeve includes a set of ridges protruding from an outer surface thereof.

Additionally or alternatively a nineteenth non-limiting example takes the form of an IRM as in the seventeenth to eighteenth non-limiting examples wherein the suture sleeve and torsion tube are configured to compress onto the lead to prevent longitudinal and rotational movement of the lead relative to the torsion tube.

Additionally or alternatively a twentieth non-limiting example takes the form of an IRM as in the thirteenth to nineteenth non-limiting examples wherein the torsion tube comprises a support structure configured to enhance a column strength of the torsion tube.

Additionally or alternatively a twenty-first non-limiting example takes the form of an IRM as in the twentieth non-limiting example wherein the support structure is absent beneath the suture sleeve.

A twenty-second non-limiting example takes the form of implanting a lead having an electrode structure in a patient comprising inserting the lead, with an IRM as in any of the thirteenth to twenty-first non-limiting examples thereon, into a patient, with a sheath covering the securing structure in the pre-deployment state, and at least partly withdrawing the sheath to deploy the securing structure to bias the electrode structure in a desired direction.

A twenty-third non-limiting example takes the form of an implantable lead for use with an implantable cardiac stimulus device, the lead comprising a lead body having a longitudinal axis extending between a proximal end and a distal end, wherein the proximal end is adapted for coupling to the implantable cardiac stimulus device, an electrode structure disposed adjacent to the distal end of the lead body, and a securing structure, having at least one flap, located near the electrode structure and configured to move from a pre-deployment state to a deployed state, wherein in the pre-deployment state the flap is compressed and in the deployed state the flap extends away from the lead body to push against a first portion of tissue of a patient causing the electrode structure to press against a second portion of tissue of the patient.

Additionally or alternatively a twenty-fourth non-limiting example takes the form of a lead as in the twenty-third non-limiting example wherein the securing structure includes a plurality of flaps radially spaced around the lead body.

Additionally or alternatively a twenty-fifth non-limiting example takes the form of a lead as in any of the twenty-third to twenty-fourth non-limiting examples wherein the at least one flap is included adjacent to the electrode structure.

Additionally or alternatively a twenty-sixth non-limiting example takes the form of a lead as in any of the twenty-third to twenty-fifth non-limiting examples wherein a first end of the at least one flap is molded to the lead body, such that a second end of the at least one flap extends away from the lead body in the deployed state.

Additionally or alternatively a twenty-seventh non-limiting example takes the form of a lead as in any of the twenty-third to twenty-sixth non-limiting examples further comprising a suture sleeve located proximal of the securing structure and including a body having a longitudinal channel to receive the lead body, and one or more channels to receive a suture thereon.

Additionally or alternatively a twenty-eighth non-limiting example takes the form of a lead as in any of the twenty-third to twenty-seventh non-limiting examples wherein the at least one flap comprises a fan shape.

Additionally or alternatively a twenty-ninth non-limiting example takes the form of a lead as in any of the twenty-third to twenty-eighth non-limiting examples wherein the at least one flap comprises a tine shape.

Additionally or alternatively a thirtieth non-limiting example takes the form of a method of implanting a lead as in any of the twenty-third to twenty-ninth non-limiting examples in a patient comprising inserting the lead into a patient with a sheath covering the securing structure in the pre-deployment state, and at least partly withdrawing the sheath to deploy the securing structure to bias the electrode structure in a desired direction.

Additionally or alternatively a thirty-first non-limiting example takes the form of a method as in any of the twenty-second or thirtieth non-limiting examples wherein the step of inserting the lead is performed by inserting the lead into a blood vessel.

Additionally or alternatively a thirty-second non-limiting example takes the form of a method as in the twenty-third non-limiting example wherein the blood vessel is an internal thoracic vein.

Additionally or alternatively a thirty-third non-limiting example takes the form of a method as in any of the twenty-second or thirtieth non-limiting examples further comprising adjusting the lead position prior to completely withdrawing the sheath by using the sheath to retract the securing structure from the deployed state.

A thirty-fourth non-limiting example takes the form of a method of implanting a lead assembly, the method comprising establishing access to a patient, inserting the lead assembly into the patient from the access, wherein the lead assembly includes a body having an outer surface and a longitudinal axis extending between a proximal end and a distal end, a lumen extending from the proximal end to the distal end along the longitudinal axis and configured to receive a positioning mechanism, a set of electrode structures, and a plurality of channels extending from the proximal end to the distal end of the body and radially spaced from one another, each of the channels being configured to receive an electrode structure, of the set of electrode structures, therein such that the body electrically isolates the electrode structure from other electrode structures, of the set of electrode structures, and enables contact between tissue of the patient and the electrode structure located in the lumen, and advancing the lead assembly to a desired location relative to a heart of a the patient using the positioning mechanism.

Additionally or alternatively a thirty-fifth non-limiting example takes the form of a method as in the thirty-fourth non-limiting example wherein the step of advancing the lead assembly to the desired location comprises advancing the lead assembly subcutaneously over a ribcage of the patient.

Additionally or alternatively a thirty-sixth non-limiting example takes the form of a method as in the thirty-fourth non-limiting example wherein the step of advancing the lead assembly to the desired location comprises advancing at least a portion of the lead assembly substernally but without contacting the heart or entering or securing to a pericardium of the heart.

Additionally or alternatively a thirty-seventh non-limiting example takes the form of a method as in the thirty-fourth non-limiting example wherein the step of advancing the lead assembly to the desired location comprises advancing at least a portion of the lead assembly in an internal thoracic vein of the patient.

Additionally or alternatively a thirty-eighth non-limiting example takes the form of a method as in the thirty-fourth non-limiting example wherein the step of advancing the lead assembly to the desired location comprises advancing at least a portion of the lead assembly in an intercostal vein of the patient.

A thirty-ninth non-limiting example takes the form of a method of using a lead assembly, the method comprising implanting the lead assembly as in the thirty-fourth non-limiting example, and selecting at least one electrode structure in a channel, of the plurality of channels, for delivery of pacing therapy to the heart of a patient, but not selecting at least one other electrode structure in the channel.

Additionally or alternatively a fourteenth non-limiting example takes the form of a method as in the thirty-ninth non-limiting example wherein the step of implanting the lead assembly at the desired location comprises implanting the lead assembly subcutaneously over a ribcage of the patient.

A forty-first non-limiting example takes the form of a method of using a lead assembly, the method comprising implanting the lead assembly as in the thirty-fourth non-limiting example, and delivering a defibrillation therapy by electrically coupling at least two of the electrodes structures together as one pole for outputting a defibrillation therapy shock.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

FIG. 1A illustrates a side-view of a channel body for use in a lead assembly;

FIG. 1B illustrates a front-view of the channel body of FIG. 1A;

FIG. 2A illustrates an exemplary embodiment of a channel body;

FIG. 2B illustrates another exemplary embodiment of a channel body;

FIG. 2C illustrates another exemplary embodiment of a channel body;

FIG. 3 illustrates a schematic block diagram of an illustrative IMD;

FIG. 4A illustrates an IMD in conjunction with a lead assembly having a set of channel bodies;

FIG. 4B illustrates the IMD and lead assembly of FIG. 4A implanted in a patient;

FIG. 5 illustrates an implantable retention mechanism (IRM) for use with an implantable lead;

FIG. 6A illustrates a top-view of an exemplary embodiment of a securing structure;

FIG. 6B illustrates a top-view of another exemplary embodiment of a securing structure;

FIG. 6C illustrates a top-view of another exemplary embodiment of a securing structure;

FIG. 6D illustrates a side-view of another exemplary embodiment of a securing structure;

FIG. 6E illustrates a side-view of another exemplary embodiment of a securing structure;

FIG. 7 illustrates a top-view of an exemplary embodiment of a suture sleeve;

FIG. 8 illustrates a an exemplary embodiment of an implantable lead;

FIG. 9 illustrates the implantable lead implanted in a patient; and

FIG. 10 is a block flow diagram for an illustrative method.

### DETAILED DESCRIPTION

The S-ICD System from Boston Scientific provides benefits to the patient including the preservation of transvenous anatomy and avoidance of intracardiac leads, which may fracture and/or may serve as conduits for infection to reach the heart. Transvenous, intracardiac leads may also occlude blood vessels going into the heart, making later placement of leads or other devices in the heart more difficult, another issue avoided with the S-ICD System. Some examples and discussion of subcutaneous lead implantation may be found in U.S. Pat. No. 8,157,813, titled APPARATUS AND METHOD FOR SUBCUTANEOUS ELECTRODE INSERTION, and US PG Publication No. 20120029335, titled SUBCUTANEOUS LEADS AND METHODS OF IMPLANT AND EXPLANT, the disclosures of which are incorporated herein by reference. Additional subcutaneous placements are discussed in U.S. Pat. No. 6,721,597, titled SUBCUTANEOUS ONLY IMPLANTABLE CARDIOVERTER DEFIBRILLATOR AND OPTIONAL PACER, and the above mentioned U.S. Pat. No. 7,149,575, the disclosures of which are incorporated herein by reference.

While many patients can be well treated with the S-ICD System, there continue to be limitations. Increased energy requirements of the S-ICD System, perceived difficulty with providing chronic bradycardia pacing, and unavailability of anti-tachycardia pacing to terminate select fast tachycardias, have created interest in alternative defibrillator and/or pacemaker placement techniques. One proposal has included a substernal placement, with a lead extending beneath the sternum from a position inferior to the lower rib margin, such as in U.S. patent application Ser. No. 15/208,682, titled SUBSTERNAL PLACEMENT OF A PACING OR DEFIBRILLATING ELECTRODE, the disclosure of which is incorporated herein by reference. Proposals for a substernal device have been referred to as extravascular, insofar as the lead does not enter or reside in the vasculature. Such devices are distinct from early generation epicardial devices in that the lead and electrode would not touch the heart or enter or be secured to the pericardium.

In human anatomy, the internal thoracic vein (ITV), which may also be referred to as the internal mammary vein, is a vessel that drains the chest wall and breasts. There are both left and right internal thoracic veins on either side of the sternum, beneath the ribs. The ITV arises from the superior epigastric vein, accompanies the internal thoracic artery along its course and terminates in the brachiocephalic vein. The ITV may make a suitable location for placement of a cardiac stimulus lead. While much of the following disclosure focuses on the use of the ITV, many of these concepts could also be applied to the internal thoracic arteries, which may sometimes be referenced as the internal mammary arteries. Some additional details related to the use of the ITV for placement of cardiac leads may be found in U.S. patent application Ser. No. 15/667,167, titled IMPLANTATION OF AN ACTIVE MEDICAL DEVICE USING THE INTERNAL THORACIC VASCULATURE, the disclosure of which is incorporated herein by reference.

Embodiments of the present invention may take the form of devices with leads sized and adapted for placement subcutaneously, in the internal thoracic vein (or a tributary thereto such as an intercostal vein), and/or in a substernal position. Other implant positions are also envisioned, such as placement in the heart and/or using other vessels such as the azygos vein.

Electrode structures may be employed to allow electrical stimulation to heart tissue and/or sense a physiological condition. Prior devices include ring electrodes for pacing and sensing, and longer coil electrodes for higher energy defibrillation. The coil electrodes, with greater surface area, can reduce impedance at the tissue interface to make therapy delivery more efficient and effective than smaller electrodes can achieve. However, coil electrodes generally allow the output current to flow in all directions, yet the therapy target resides on only one side of the electrode. Also, the large surface area of a standard coil electrode can allow it to capture more sensing signals, particularly muscle and motion noise, than smaller ring electrodes. An approach which allows directional selectivity and combines the advantages of smaller electrodes with those of larger electrodes is desired.

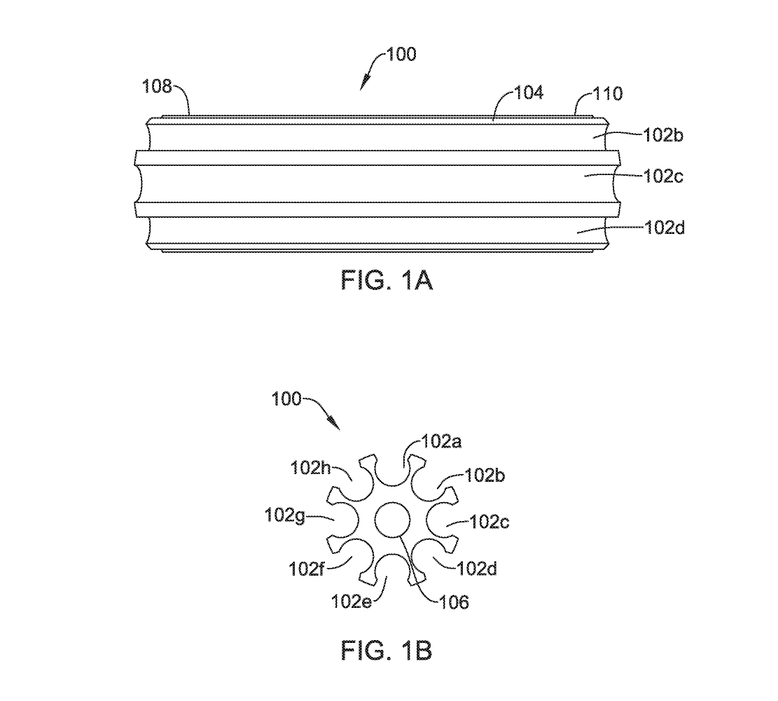

FIGS. 1A-1B depict an example of a channel body 100 which may be used in a lead assembly for use in conjunction with an implantable medical device (IMD) in order to detect and/or treat cardiac abnormalities. FIG. 1A depicts a side-view of the channel body 100 and FIG. 1B depicts a front-view of the channel body 100. In certain embodiments, a size of the channel body 100 may range between about 4 to about 12 French, for example, 4, 5, 6, 7, 8, 9, 10, 11 or 12 French, or larger or smaller.

As shown, the channel body 100 may include channels 102A-102H that extend from a proximal end 108 of the channel body 100 to a distal end 110 of the channel body 100 and are configured to receive and hold, encompass, contain, and/or secure an electrode structure, therein. In some cases, the diameter of the channels 102A-102H may range between 0.01″ and 0.015″, or smaller or larger. In other cases, the diameters may depend on the number of channels located on the channel body 100. A channel body that has only one or two channels may have channel diameters that are larger than those of a channel body having six to eight channels, for example.

In certain embodiments, an outer surface 104 of the channel body 100 may have gaps or slits that extend from the outer surface 104 to the channels 102A-102H. In some cases, the gaps may allow electrode structures in the channels 102A-102H to make contact with the tissue of the patient when the channel body 100 is positioned at the desired location within the patient. The combination of the channels with the gaps or slits may be configured to retain the electrode structures in the channels against movement laterally out of the channels. In other examples, a conductive surface, such as a conductive or porous polymer, may extend over the channels to retain the electrode structures therein.

According to various embodiments, the electrode structures can be secured relative to the channel body 100 but exposed to the tissue surrounding the channel body 100. In some cases, the electrode structures may be a wire, cable, or coil electrode element and may be in electrical communication with operational circuitry (not shown in FIGS. 1A and 1B) of the IMD. The electrode structures may also be ring electrodes or partial ring electrodes (half, third, two-third, etc.) on or wrapped about a conductor. In certain embodiments, the electrode structures may be disposed on conductors that are connected to the IMD through connecting wires. In examples where the conductors include one or more electrode structures, the electrode structures may in some cases be disposed at different parts along the conductors and several conductors may be disposed inside several different channels 102A-102H. In some cases, the increase in the number of electrode structures may allow for efficient sensing of cardiac electrical activity, delivery of electrical stimulation (e.g., directed electrical stimulation), and/or communication with an external medical device. The electrode structures can be made up of one or more biocompatible conductive materials, such as various metals or alloys that are known to be safe for implantation within a human body. In some instances, the electrode structures connected within the channels 102A-102H may have an insulative portion that electrically isolates the electrode structures from adjacent electrode structures, particularly along locations of the device where the channel body is absent.

In some instances, the channels 102A-102H may be radially spaced from one another around the channel body 100 and portions and/or the entire channel body 100 may be comprised of materials, for example dielectric materials, that electrically insulate the electrode structures from one another. In some cases, the channel body 100 may provide galvanic isolation such that current flow or a direct conduction path is prevented between the electrode structures from one channel to another.

According to various embodiments, the channel body 100 may also have a protective layer around the outer surface 104 and on an outer surface of each of the channels 102A-102H. In certain embodiments, the channel body 100 may be sealed by a biocompatible protective layer. The biocompatible protective layer may include any suitable material including, for example, titanium and its alloys, noble metals and their alloys, biograde stainless steels, cobalt-based alloys, tantalum, niobium, titanium-niobium alloys, Nitinol, MP35N (a nickel-cobalt-molybdenum alloy), alumina, zirconia, quartz, fused silica, biograde glass, silicon, and some biocompatible polymers. These are just examples. In some cases, the protective layer may provide a barrier from the body including cells, proteins, platelets, and/or other biological and/or chemical agents. In some instances, the protective layer may allow energy transfer between electrode structures located in the channels 102A-102H. In some cases, the protective layer provides a hermetic seal. Alternatively, the protective layer may be porous to body fluid such as blood or blood plasma. In some cases, portions and/or the entire channel body 100 may be composed of a corrosion resistant material, for example, gold, silver, stainless steel, etc. and not have a protective layer. Materials may be selected, or coatings provided, to prevent thrombi from forming in or on the electrode structures, channels, channel body, and/or protective layer.

As shown in FIG. 1B, in some cases, the channel body 100 may also include a lumen 106 that extends from the proximal end 108 to the distal end 110 and is configured to receive a positioning mechanism, such as a guidewire or a stylet, for example. In some cases, the diameter of the lumen 106 may range between 0.01″ and 0.018″, however, this is only an example. In other embodiments, the diameter of the lumen 106 may be larger or smaller. In some instances, the guidewire or stylet may be inserted into the lumen 106 at the proximal end 108 and advanced to the desired location in the patient. In some examples, the guidewire or stylet is preloaded in the channel body 100 and both are introduced at the same time until the channel body 100 is at the desired location. The guidewire or stylet may be deflectable or steerable. In some embodiments, the guidewire or stylet may preferably be stiff or curved. In other embodiments, the guidewire or stylet may be preferably flexible. In some examples, at least two guidewires or stylets may be used, a first more flexible and steerable guidewire or stylet to obtain initial access to a first location in the patient and a second, stiffer guidewire or stylet to reach the second, desired, location in the patient.

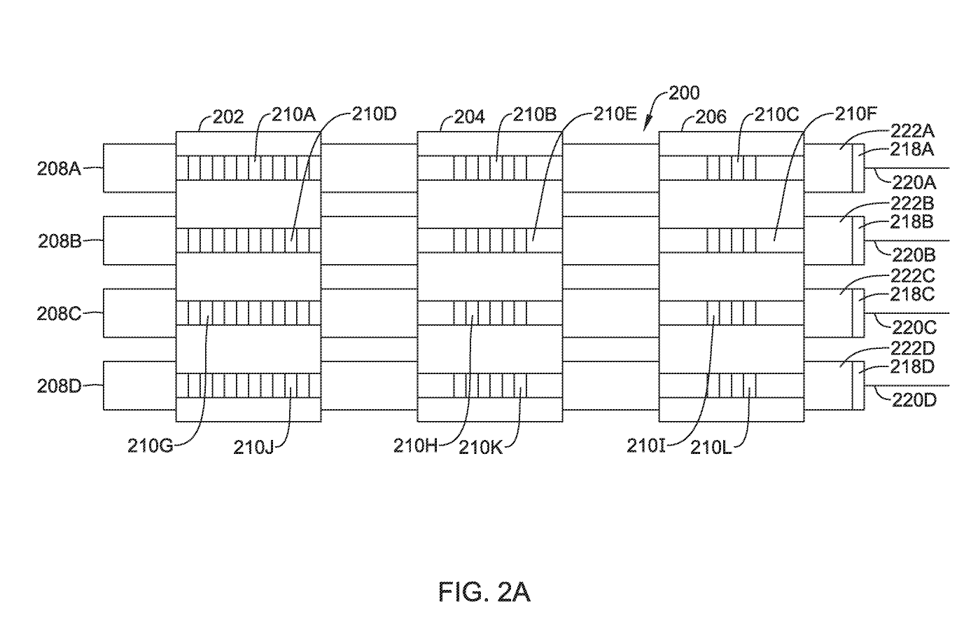

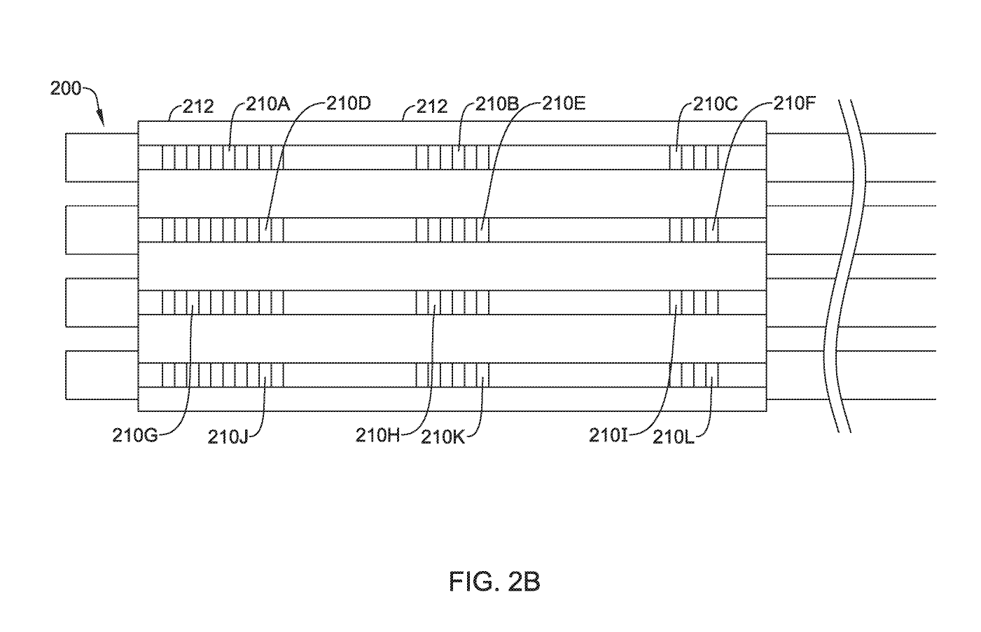

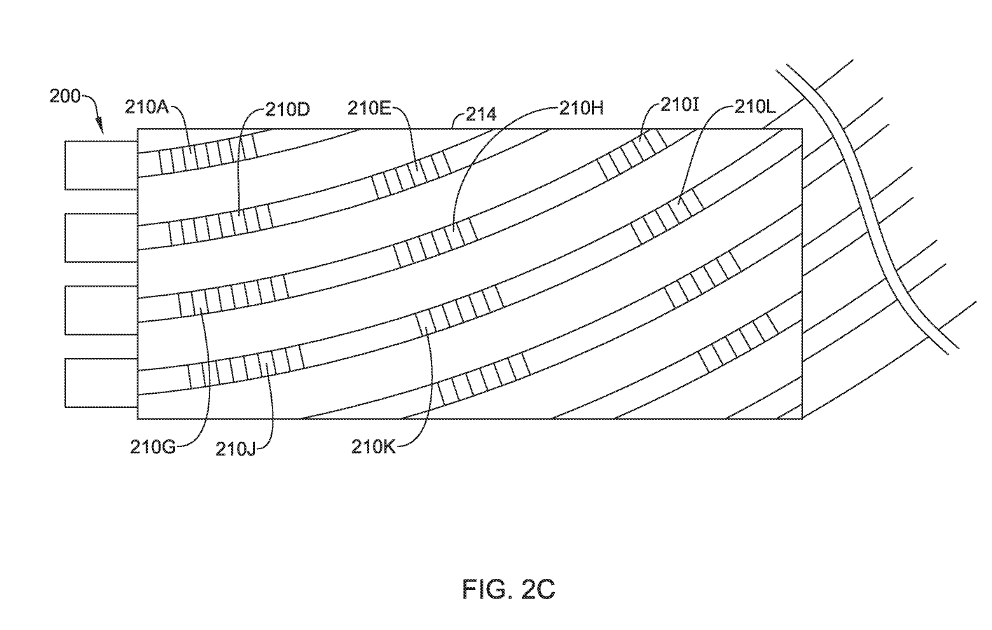

As depicted in FIGS. 2A-2C, a channel body may be used in conjunction with an IMD that may include a lead assembly 200. The lead assembly 200 may include conductors 208A-208D connected at proximal ends 222A-222D by connectors 218A-218D to electrical wires 220A-220D. The electrical wires 220A-220D may extend from the conductors 208A-208D to the IMD and may conduct electrical signals between electrode structures 210A-210L, contained on the conductors 208A-208D, and operational circuity (not in FIGS. 2A-2C) of the 1 MB. In some examples, the lead assembly 200 may be implanted on, within, or adjacent to a heart of a patient and the electrodes structures 210A-210L may be positioned at various locations on the conductors 208A-208D, and in some cases at various distances from the 1 MB. Some conductors 208A-208D may only include a single electrode, while other conductors 208A-208D may include multiple electrodes structures. Generally, the electrode structures 210A-210L are positioned on the conductors 208A-208D such that when the conductors 208A-208D are implanted within the patient, one or more of the electrode structures 210A-210L are positioned to perform a desired function. In some cases, the one or more of the electrode structures 210A-210L may be in contact with the patient's cardiac tissue. In some cases, a lead assembly 200 including the electrode structures 210A-210L may be positioned entirely subcutaneously and outside of the patient's heart. In some cases, the electrode structures 210A-210L may conduct electrical signals to the conductors 208A-208D, e.g. signals representative of intrinsic cardiac electrical activity. The conductors 208A-208D may, in turn, conduct the received electrical signals to the operational circuitry of the IMD. In some cases, the IMD may generate electrical stimulation signals, and the conductors 208A-208D may conduct the generated electrical stimulation signals to the electrode structures 210A-210L. The electrode structures 210A-210L may then conduct the electrical signals and deliver the signals to the patient's heart (either directly or indirectly).

Referring to FIG. 2A, in certain embodiments, the lead assembly 200 may be inserted into the channels of multiple channel bodies 202, 204, and 206 such that the electrode structures 210A-210L are disposed in the channels and exposed thereby for contact with the tissue of the patient. As shown, the channels may extend in a longitudinal direction along the channel bodies 202, 204, and 206. In some cases, the length of the channel bodies 202, 204, and 206 may be less than the length of the lead assembly 200. In various embodiments, the length of the channel bodies 202, 204, and 206 may be less than ½, ⅓, ¼, etc. than the length of the lead assembly 200.

Referring to FIG. 2B, in certain embodiments, the lead assembly 200 may be inserted into the channels of channel body 212. Similar to FIG. 2A, the electrode structures 210A-210L are disposed in the channels and exposed thereby for contact with the tissue of the patient. As shown, the channels may extend in a longitudinal direction along the channel body 212. In some cases, the length of the channel body 212 may be less than the length of the lead assembly 200. In various embodiments, the length of the channel body 208 may be 60%, 70%, 80%, 90%, etc. the length of the lead assembly 200.

Referring to FIG. 2C, in certain embodiments, the lead assembly 200 may be inserted into the channels of channel body 214. The channel body 214 may be similar to the channel body 212 (e.g., similar lengths and the electrode structures 210A-210L are disposed in the channels and exposed thereby for contact with the tissue of the patient). However, the channels of the channel body 214 may extend helically about the channel body 214. In this instance, the conductors 208A-208D may be flexible and capable of bending in the helical direction without being damaged.

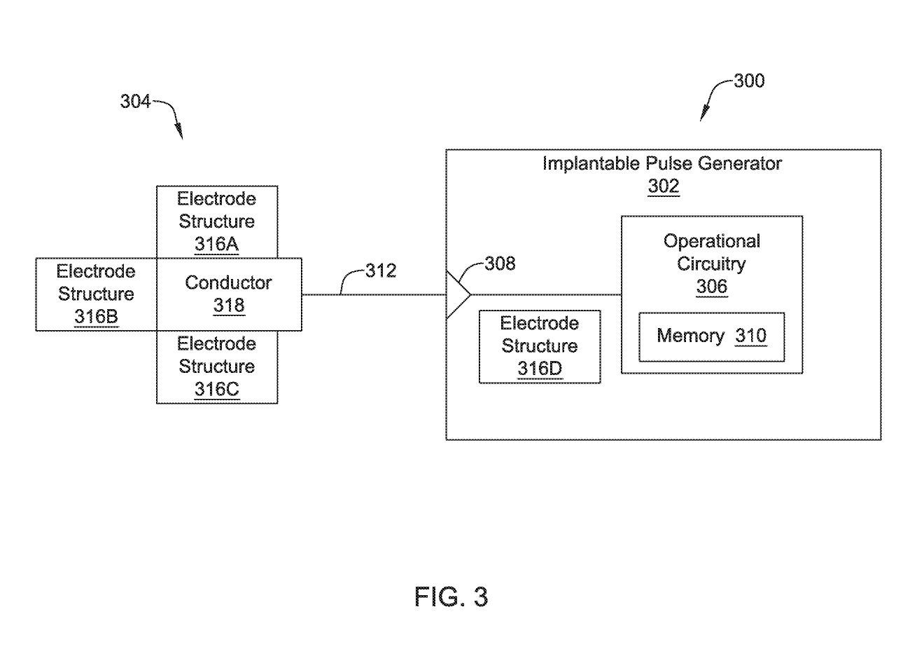

FIG. 3 is a schematic block diagram of an illustrative IMD 300. In some cases, the illustrative IMD 300 may include an implantable pulse generator 302 and a lead assembly 304. As shown, the implantable pulse generator 302 may include operational circuitry 306, an electrode structure 316D, and a port 308. In some examples, the operational circuitry 306 may include a pre-programmed chip, such as a very-large-scale integration (VLSI) chip and/or an application specific integrated circuit (ASIC). In such embodiments, the chip may be pre-programmed with control logic in order to control the operation of the IMD 300. By using a pre-programmed chip, the operational circuitry 306 may use less power than other programmable circuits (e.g. general purpose programmable microprocessors) while still being able to maintain basic functionality, thereby potentially increasing the battery life of the IMD 300. In other examples, the operational circuitry 306 may include a programmable microprocessor. Such a programmable microprocessor may allow a user to modify the control logic of the IMD 300 even after implantation, thereby allowing for greater flexibility of the IMD 300 than when using a pre-programmed ASIC. In some examples, the operational circuitry 306 may further include a memory 310, and the operational circuitry 306 may store information on and read information from the memory 310. In other examples, the operational circuitry 306 may include a separate memory (not shown) that is in communication with the operational circuitry 306, such that the operational circuitry 306 may read and write information to and from the separate memory

In various embodiments, the port 308 may be electrically coupled to the operational circuitry 306 and configured to receive a connector 312 that may electrically couple conductor 318, to the operational circuitry 306. The port 308 may be provided on a header attached to a hermetically sealed canister/housing with electrical connections via a feedthrough assembly. Furthermore, the conductor 318 may be electrically coupled to electrode structures 316A-316C, therefore, the connector 312 may electrically connect the lead assembly 304 to the operational circuitry 306. In some cases, the electrode structures 316A-316D may be sensing and/or pacing electrodes, capable of being positioned against or may otherwise contact a patient's tissue to provide electro-therapy and/or sensing capabilities.

For example, the IMD 300 may be an implantable cardiac pacemaker (ICP) and as discussed above, the operational circuitry 306 may include pre-programmed VLSI chip, an ASIC, or a programmable microprocessor. Regardless of whether the operational circuitry 306 is located on a pre-programmed chip or a programmable microprocessor, the operational circuitry 306 may be programmed with logic where the operational circuitry 306 can select a first electrode (e.g., electrode structure 316A) and select a second electrode (e.g., electrode structure 316B or 316D) as an opposing electrode. The operational circuitry 306 may then use electrode structure 316A and 316B or 316D to sense intrinsically generated cardiac electrical signals and determine, for example, one or more cardiac arrhythmias based on analysis of the sensed signals. The operational circuitry 306 may then be configured to deliver defibrillation, cardioversion, CRT, ATP therapy, bradycardia therapy, and/or other therapy types via the electrode structures 316A and 316B or 316D. In another example, the IMD 300 may be an implantable cardioverter-defibrillator (ICD). In such examples, the operation circuitry 306 may also be configured to link electrode structures 316A and 316B and select electrode structure 316D as an opposing electrode. The operational circuitry may then use the electrode structure 316A, 316B, and 316D to sense cardiac electrical signals, determine occurrences of tachyarrhythmias based on the sensed signals, and may be configured to deliver defibrillation therapy in response to determining an occurrence of a tachyarrhythmia via the electrode structures 316A. 316B and 316D. In other examples, the IMD 300 may be a subcutaneous implantable cardioverter-defibrillator (S-ICD). In examples where the IMD 300 is an S-ICD, the lead assembly 304 may be implanted subcutaneously without entering or contacting the heart.

In various embodiments, the operational circuitry 306 and therefore, the implantable pulse generator 302, may have electrode activation and assessment programmability to facilitate efficient optimization of electrode activation to deliver optimized therapy and/or to select electrode pairings for sensing cardiac activity. In the event of a capture or defibrillation failure, the implantable pulse generator 302 may have automatic electrode switching capability to correct performance issues.

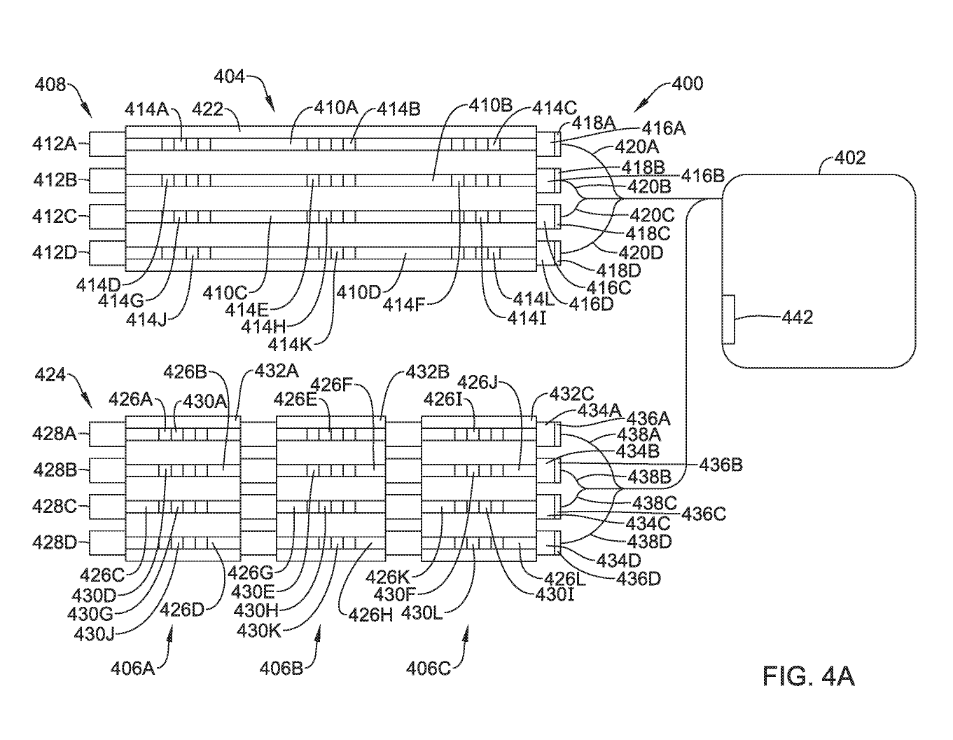

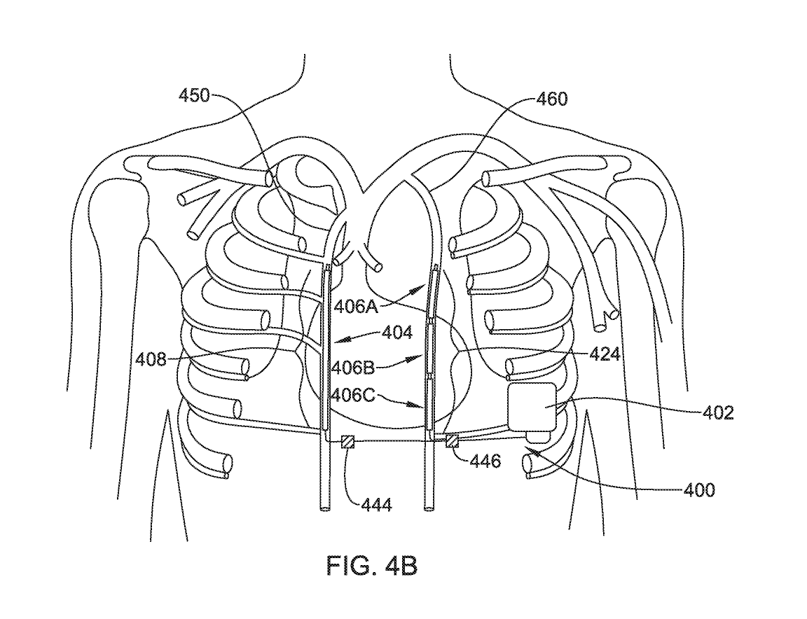

FIG. 4A provides a schematic of an IMD 400 in conjunction with a first channel body 404 and a set of second channel bodies 406A-406C. As shown, the first channel body 404 holds a first lead assembly 408. In particular, individual conductors 412A-412D are inserted into and held by channels 410A-410D. Electrode structures 414A-414L are disposed on the conductors 412A-412D and exposed by gaps in an outer surface 422 of the channel body 404 to allow the electrode structures 414A-414L to make contact with tissue of a patient. According to various embodiments, the electrode structures 414A-414L may be different electrodes (e.g., wire, cable or coil electrodes). As shown, the channels 410A-410D extend in a longitudinal direction along the channel body 404. In this case, the length of the channel body 404 is about 90% of the length of the lead assembly 408. In other examples, the channel body 404 may form a shorter length of the lead assembly 408, for example, approximately four to ten centimeters of a forty to sixty centimeter long lead assembly 408. In various embodiments, the conductors 412A-412D may be connected at proximal ends 416A-416D by connectors 418A-418D to electrical wires 420A-420D. The electrical wires 420A-420D may extend from the conductors 412A-412D to an implantable pulse generator (IPG) 402 and may conduct electrical signals between electrodes 414A-414L and the IPG 402. In certain embodiments, the IPG 402 also includes an electrode structure 442.

As further depicted in FIG. 4A, the second set of channel bodies 406A-406C hold a second lead assembly 424. In particular, individual conductors 428A-428D are inserted into and held by channels 426A-426L. Electrodes structures 430A-430L are disposed on the conductors 428A-428D and exposed by gaps in outer surfaces 432A-432C of the channel bodies 406A-406C to allow the electrode structures 430A-430L to make contact with the tissue of the patient. According to various embodiments, the electrode structures 430A-430L may be different electrodes (e.g., wire, cable or coil electrodes). As shown, the channels 426A-426L extend in a longitudinal direction along the channel bodies 406A-406C. In this case, the lengths of the channel bodies 406A-406C are less than half of the length of the lead assembly 424. In various embodiments, the conductors 428A-428D may be connected at proximal ends 434A-434D by connectors 436A-436D to electrical wires 438A-438D. The electrical wires 438A-438D may extend from the conductors 428A-428D to the IPG 402 and may conduct electrical signals between the electrode structures 430A-430L and the IPG 402. According to various embodiments, the second set of channel bodies 406A-406C may be arranged along the length of the lead assembly 424 to provide programmable sensing and/or electro-therapy from either channel body 406A, 406B, or 406C.

In various embodiments, a “conductor” (e.g., conductors 412A-412D or 428A-428D) may be a single conductor with just one electrical connection from a proximal end to a distal end of an electrode structure, or it may be multiple conductors to allow, for example, separately addressing different electrode structures along its length to allow, for example, an electrode structure on conductor 428A disposed in channel body 406A to be separately addressed from an electrode structure disposed in channel body 406B. Separate proximal contacts may be provided for each such separately addressable electrode structure on a given conductor.

FIG. 4B shows implantation of the first channel body 404 in the right internal thoracic vein (ITV) 450 and the set of second channel bodies 406A-406C in the left ITV 460. A first suture sleeve 444 secures the first channel body 404 at first location in the right ITV 450 and a second suture sleeve 446 secures the set of second channel bodies 406A-406C at a second location in the left ITV 460. The IPG 402 may be implanted in the left axilla. As stated herein, in certain embodiments, the IMD 400 may provide pacing output or obtain sensing signals by selecting a first electrode (e.g., electrode structure 430A, from FIG. 4A) and select a second electrode (e.g., electrodes structure 430B, 430K or 442, from FIG. 4A) as an opposing electrode. The IMD 400 may then be configured to deliver CRT, ATP therapy, bradycardia therapy, and/or other therapy types via the electrode structures 430A and 430B, 430K, or 442. A first electrode combination may be used for both sensing and therapy, though in other examples, first and second different combinations are used for sensing and therapy, respectively. In another example, the IMD 400 may be an ICD. In such examples, the ICD may be configured to link first electrodes (e.g., 430A and 430B) and select a second electrode (e.g., 430K or 442) as an opposing electrode. The ICD may then use the electrode structures 430A, 430B, and 430K or 442 to sense cardiac electrical signals, determine occurrences of tachyarrhythmias based on the sensed signals, and deliver defibrillation therapy in response to determining an occurrence of a tachyarrhythmia via the electrode structures 430A. 430B and 430K or 442. In other examples, the IMD 400 may be a subcutaneous implantable cardioverter-defibrillator (S-ICD). These are only a few examples. Other electrode structures and/or multiple electrode structures and electrode structure configurations may be selected to deliver pacing and/or defibrillation therapy. For instance, in certain embodiments, operational circuitry (e.g., operational circuitry 306, from FIG. 3) may be configured to measure the impedances between the electrode structures 414A-414L (from FIG. 4A) and 430A-430L and determine which electrode structures to use for delivering pacing therapy based on the measured impedances. For example, the impedance measurements could be used to identify which electrode structures 414A-414L and 430A-430L have the best tissue contact and the electrode structures 414A-414L and 430A-430L that have the best tissue contact may be activated. Alternatively or additionally, pacing thresholds and sensing measurements may be taken to determine and select the electrode structures 414A-414L and 430A-430L that work effectively and efficiently individually or in combination with other electrode structures. For example, in some cases, all the electrode structures (i.e., 414A, 414E, and 414I) from channel 410A and all the electrode structures (i.e., 430A, 430E, and 430I) from channel 426A may be activated to create a high surface area defibrillation electrode.

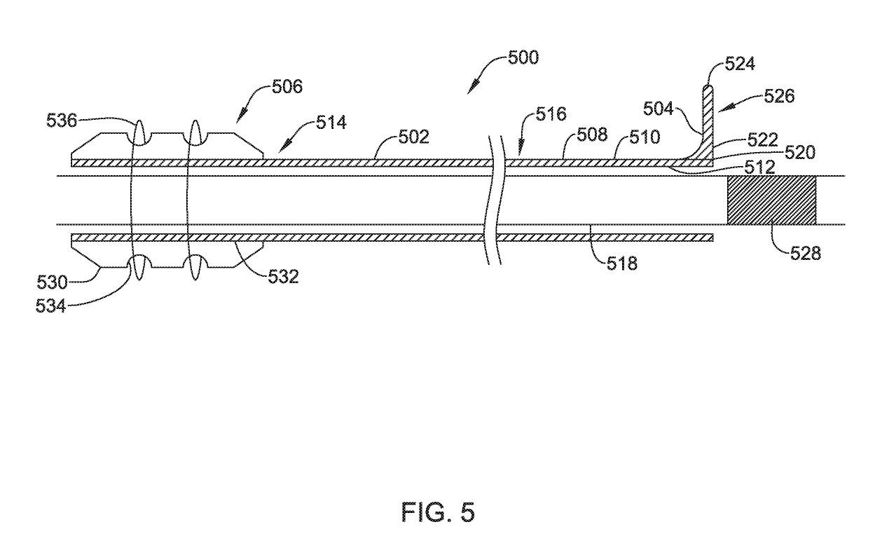

FIG. 5A provides a schematic of an implantable retention mechanism (IRM) 500 in conjunction with an implantable lead 518. As shown, the IRM may include a torsion tube 502, a securing structure 504, and a suture sleeve 506. According to various embodiments, the torsion tube 502 may extend from a proximal end 514 to a distal end 516 and may comprise a support structure configured to enhance a column strength of the torsion tube 502. The torsion tube 502 may be a single layer or multiple layer structure.

For example, in some cases, the torsion tube 502 may include a support member 510, an outer liner 508, and an inner liner 512. The support member 510 may include a plurality of filaments or wire strands braided together forming a plurality of crossing points or intersections. In some instances, the support member 510 may be formed from, for example, stainless steel, such as high tensile stainless steel, or other materials, including metals and metal alloys, such as tungsten, gold, titanium, silver, copper, platinum, palladium, iridium, ELGILOY nickel-cobalt alloys, cobalt chrome alloys, molybdenum tungsten alloys, tantalum alloys, titanium alloys, nickel-titanium alloys (e.g., nitinol), etc.

From herein the term “support member” refers to tubular constructions in which metallic or non-metallic wire strands are in a wound, braided or woven fashion as they cross to form a tubular member defining a lumen formed in at least a portion of the torsion tube 502. The support member 510 may be made up of a suitable number of wire strands, such as six, eight, twelve, sixteen, twenty-four, twenty-eight, etc. and the support member 510 may be formed through any conventional technique known by those skilled in the art. A support member may also take the form of a hypotube or cut hypotube.

In various embodiments, the inner liner 512, may be a tubular sleeve, formed of a polymeric material, disposed within the lumen formed by the support member 510, thus defining the inner lumen of the torsion tube 502. The lumen defined by the inner liner 512 may provide passage to the implantable lead 518. In some cases, the inner liner 512 is formed from a lubricious polymer, such as a fluorocarbon (e.g., polytetrafluoroethylene (PTFE)), a polyamide (e.g., nylon), a polyolefin, a polyimide, or the like). Additional polymeric materials which may make up the inner liner 512 include polyethylene, polyvinyl chloride (PVC), ethyl vinyl acetate (EVA), polyethylene terephthalate (PET), and their mixtures and copolymers. Another useful class of polymers is thermoplastic elastomers, including those containing polyesters as components. For example, the inner liner 512 may be formed by extruding a rigid thermoplastic elastomer polymer. The inner liner 512 can be dimensioned to define the lumen having an appropriate inner diameter to accommodate its intended use.

According to various embodiments, the torsion tube 502 further includes the outer liner 508. In some cases, a polymeric material may be extruded over the top of the inner liner 512 and the support member 510 to form the outer liner 508. In some cases, the polymeric material may be heat shrunk over the top of the inner liner 512 and the support member 510 to form the outer liner 508. In these embodiments, the support member 510 may become embedded within the outer liner 508 and the outer liner 508 may bond with the inner liner 512. The outer liner 508 can be composed of a variety of materials, such as soft thermoplastic material, polyurethanes, silicone rubbers, nylons, polyethylenes, fluorinated hydrocarbon polymers, and the like. In some embodiments, the outer liner 508 may also be of a member selected from a more flexible material such as low density polyethylene (LDPE), polyvinylchloride, THV, etc. and other polymers of suitable softness or a modulus of elasticity. In some cases, the outer liner 508 may have an inner diameter that is slightly greater than the inner liner 512 and accommodates the thickness of the support member 510. In some cases, the inner liner 512 and the outer liner may be formed of the same material and the liners 508 and 512 may be extruded onto the support member 510.

In various embodiments, the securing structure 504 may be located adjacent to the distal end 516 of the torsion tube 502 and may be configured to move from a compressed, pre-deployment state, to an extended, deployed state. In some cases, the securing structure 504 may have a flap 526 that is a formed, single-piece, with the torsion tube 502. In some cases, the flap 526 may have a proximal end 522 attached to the distal end 516 of the torsion tube 502 in any suitable manner, which may include hinges, screws, pins and/or any other suitable fastener. In some cases, the proximal end 522 of the flap 526 may be molded to the distal end 516 of the torsion tube 502 so that a joint 520 is formed at the distal end 516. In some cases, the joint 520 may be configured to pivot so that a distal end 524 of the flap 526 moves, retracts, or compresses towards the torsion tube 502 to a compressed state. In some cases, the joint 520 may be further configured to pivot so that the distal end 524 of the flap 526 moves, swings, or extends away from the torsion tube 502 to an extended state.

In some cases, the securing structure 504 may be in the compressed state before and during deployment of the IRM 500, and in the extended state when the IRM 500 is deployed. For example, the IRM 500 may initially be located beneath a sheath (not shown). Beneath the sheath, the securing structure 504 may be compressed or retracted by an inner-wall of the sheath. Once IRM 500 has been positioned at a desired implantation site of a patient, the sheath may be partly or wholly withdrawn from the IRM 500. Once, the sheath has been withdrawn, the inner-wall may no longer compress or retract the securing structure 504 allowing the securing structure 504 to extend to the deployed state. In the extended, deployed state, the distal end 524 of the flap 526 may be configured to push against or away from a first portion of tissue of the patient. By pushing against the first portion of tissue, the securing structure 504 causes an electrode structure 528, disposed on the implantable lead 518, to press against a second portion (e.g., separate from the first portion, opposite the first portion, etc.) of tissue of the patient. In some cases, this may allow the electrode structure 528 to stay in contact with or increase contact with the second portion of tissue which may benefit the capability of the electrode structure to provide cardiac sensing and/or electro-therapy.

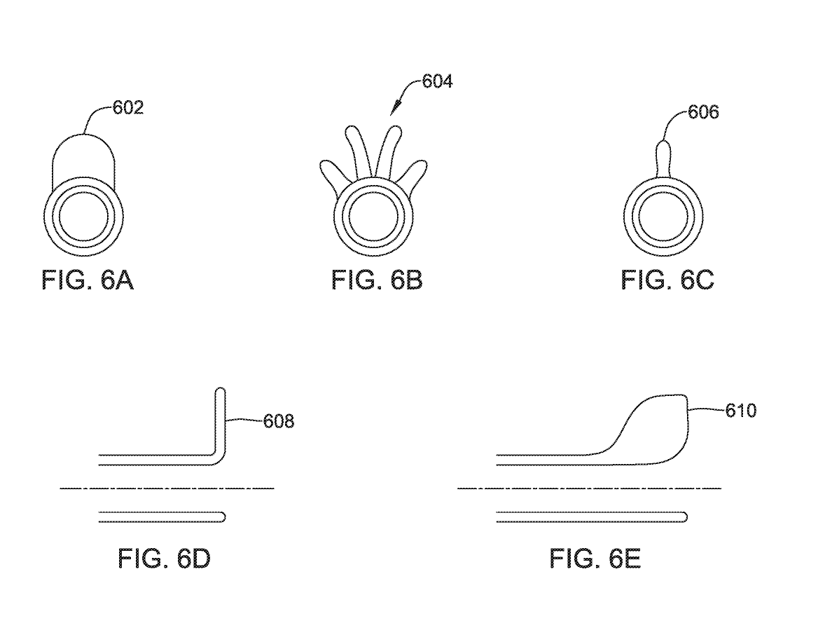

With reference now to FIGS. 6A-6E, exemplary embodiments of the securing structure are depicted. In various embodiments, the securing structure may be comprised of the same materials as discussed in regard to the braided support 510, the outer liner 508, and the inner liner 512. In some cases, the securing structure may be formed as single-piece with the torsion tube 502. In some cases, the securing structure may be molded to the torsion tube 502 or attached to the torsion tube in any suitable manner, which may include hinges, screws, pins and/or any other suitable fastener. According to various embodiments, the securing structure may be arranged to enable the IRM 500 to be collapsed by a sheath to a pre-deployed state to allow it to be carried by a positioning mechanism (e.g., a delivery catheter) and navigated to a deployment site (e.g., a blood vessel) where it can be released. Upon release, the securing structure may expand such that a flap of the securing structure may engage or push against a wall of the blood vessel to secure it in place. In certain embodiments, a lead may be located within a lumen of the torsion tube 502 and an electrode structure may be disposed on the lead in a manner that when the flap pushes against the wall, the electrode structure is forced or presses against an opposing wall of the blood vessel. In this manner, the securing structure allows the electrode structure to have sufficient contact with the blood vessel wall to enable sensing and/or electro-therapy.

As seen in FIG. 6A, flap 602 may be fan shaped and as seen in FIGS. 6B and 6C, flaps 604 and 606 may be tine shaped. Furthermore, the flaps 602, 604, and 606 have rounded, distal ends. In other embodiments, the distal ends may be square, pointed, convex, etc. These are just some examples of shapes the distal ends of the flaps may possess. In certain embodiments, as depicted in FIG. 6B, the securing structure may have multiple flaps 604 that are radially spaced around the torsion tube 502. In some cases, the flaps 604 are limited to one side of the securing structure or torsion tube 502 so the flaps 604 can push against the wall of the blood vessel and push the electrode structure against the opposing wall of the blood vessel. In some cases, the flaps 604 are spaced apart to minimize adverse obstruction of blood flow through the blood vessel. In some instances, as depicted in FIGS. 6A and 6C, there may be a single flap 602 and 606 of varying width. In some cases, the flaps 602, 604, and 606 are positioned to allow the electrode structure to be fully exposed and in contact with the blood vessel. In some cases, as stated herein, the flaps 602, 604, and 606 may be foldable for insertion through a small incision and delivery to a desired location.

As seen in FIG. 6D, a profile of flap 608 may be tine shaped. As seen in FIG. 6E, a profile of flap 610 may be tine shaped. In other embodiments, the shapes of the profiles of the flaps may be rounded, square, pointed, convex, etc. These are just some examples. Moreover, as depicted in FIGS. 6D and 6E, the flaps 608 and 610 may extend to a deployed state to press the electrode structure against the tissue of a patient.

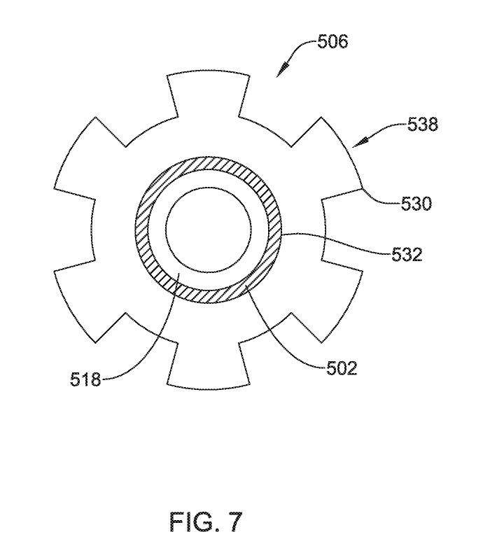

Turning back to FIG. 5, in various embodiments, the suture sleeve 506 may be located adjacent to the proximal end 514 of the torsion tube. In exemplary embodiments, the suture sleeve 506 may be formed from an elastic, biocompatible alloy capable of forming stress induced martensite (SIM). Nitinol (TiNi) is an example of such materials. The suture sleeve 506 may include an inner bore 532 extending in the longitudinal direction and as best seen in FIG. 7, may substantially surround the torsion tube 502. The suture sleeve 506 may also include a set of ridges 538 on an outer surface that can assist in securing the IRM 500 and the lead 518 at a desired location of the patient (e.g., a blood vessel). Turning again to FIG. 5, the outer surface 530 of the suture sleeve 506 may also have a plurality of circumferentially extending suture channels or grooves 534. In some cases, sutures 536 may be aligned to the suture channels 534 and tightened about the suture sleeve 506 to secure the torsion tube 502 of the IRM 500 to the lead 518. In certain embodiments, the sutures 536 may be tightened about the suture sleeve 506 and the suture sleeve 506 may compress, or collapse, from an open state to a closed state. As the suture sleeve 506 transitions from the open state to the closed state, the inner bore 532 may decrease in effective diameter. In some embodiments, the inner bore 532 may abut against the torsion tube 502, causing the inner liner 510 of the torsion tube 502 to abut against the lead 518, when the suture sleeve 506 is in either the open state, or in a partially closed state. In this configuration, the transition of the suture sleeve 506 to the closed state results in compression of the suture sleeve 506 against the torsion tube 502, and the torsion tube 502 against the lead 518. In some cases, when the suture sleeve 506 is in the closed state, the suture sleeve 506 may prevent longitudinal and rotational movement of the lead 518 relative to the torsion tube 502.

While some examples show the torsion tube 502 including a suture sleeve 506, in other examples, the suture sleeve 506 may be provided separately and omitted form the torsion tube 502. In an example, the torsion tube 502 may become affixed in place on a lead by tying down the suture sleeve. In other examples, the torsion tube 502 may be affixed on a lead by pressure applied by the tine 526 (FIG. 5), and/or flap structure 602, 604, 606 (FIG. 6A, 6B, 6C) upon deployment thereof.

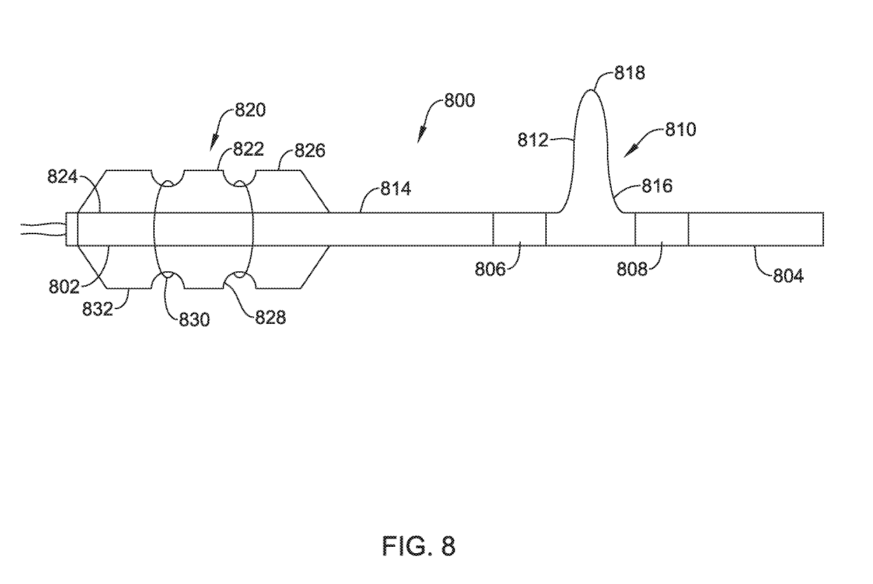

FIG. 8 depicts an implantable lead 800 for use with an implantable cardiac stimulus device. The lead 800 may be configured and operate similar to the lead assembly 200 explained in regard to FIGS. 2A-2C. In particular, the lead 800 may have a lead body 814 that extends from a proximal end 802 to a distal end 804 and the proximal end 802 may be adapted for coupling the lead 800 to the implantable cardiac stimulus device. In some examples, the lead 800 may be implanted on, within, or adjacent to a heart of a patient and electrode structures 806 and 808 may be positioned at various locations on the lead body 814, and in some cases, at various distances from the implantable cardiac stimulus device. In some cases, the lead 800 may only include a single electrode structure, while in other cases, the lead 800 may include multiple electrode structures.

Generally, the electrode structures 806 and 808 may be positioned on the lead body 814 such that when the lead 800 is implanted within the patient, one or more of the electrode structures 806 and 808 are positioned to perform a desired function. In some cases, the one or more of the electrode structures 806 and 808 may be in contact with the patient's cardiac tissue. In some cases, the one or more of the electrode structures 806 and 808 and/or the entire lead 804 may be positioned subcutaneously and outside of the patient's heart. In some cases, the electrode structures 806 and 808 may conduct intrinsically generated electrical signals to the lead 800, e.g. signals representative of intrinsic cardiac electrical activity. The lead 800 may, in turn, conduct the received electrical signals to the implantable cardiac stimulus device. In some cases, the implantable cardia stimulus device may generate electrical stimulation signals, and the lead 800 may conduct the generated electrical stimulation signals to the electrode structures 806 and 808. The electrode structures 806 and 808 may then conduct the electrical signals and deliver the signals to the patient's heart (either directly or indirectly).

According to various embodiments, the lead 800 may also include a securing structure 810 similar to the securing structure 504 from FIG. 5. In some cases, the securing structure 810 may include a flap 812 adjacent to the electrode structures 806 and 808. In some cases, the flap 812 may be formed as a single-piece with the lead body 814. In some cases, the flap 812 may have a proximal end 816 molded to the lead body 814 such that a distal end 818 of the flap extends away from the lead body 814 when in a deployed state. In some embodiments, there may be a single flap 812. In other embodiments, there may be multiple flaps that are radially spaced around the lead body 814. In some cases, the flaps are limited to one side of the lead body 814 so the flaps can push against a wall of the blood vessel and push the electrode structures 806 and 808 against an opposing wall of the blood vessel. In some cases, the flaps are spaced apart to minimize adverse obstruction of blood flow through the blood vessel. In some cases, the flap 812 may be positioned to allow the electrodes structures 806 and 808 to be fully exposed and in contact with the blood vessel. In some cases, as stated herein, the flap 812 may be foldable for insertion through a small incision and delivery to a desired location. In certain embodiments, the one or more flaps 812 may have a fan shape, in other embodiments, the one or more flaps 812 may have a tine shape. However, these are only examples and the one or more flaps 812 may take on any desirable shape or shapes.

According to various embodiments, the lead 800 may also include a suture sleeve 820 similar to the suture sleeve 506 from FIG. 5. In some cases, the suture sleeve 820 may be located proximal of the securing structure 810 and have a body 822 with an inner bore 824 extending in the longitudinal direction that is configured to substantially surround the lead body 814. The suture sleeve 820 may also include a set of ridges 832 on an outer surface 826 that can assist in securing the lead 800 at a desired location of the patient (e.g., a blood vessel). In addition, the outer surface 826 of the suture sleeve 820 may have a plurality of circumferentially extending suture channels or grooves 828. In some cases, sutures 830 may be aligned to the suture channels 828 and tightened about the suture sleeve 820 to secure the suture sleeve 820 to the lead 800. In some cases, when the suture sleeve 820 is in a closed state, the suture sleeve 820 may prevent longitudinal and rotational movement of the lead 800.

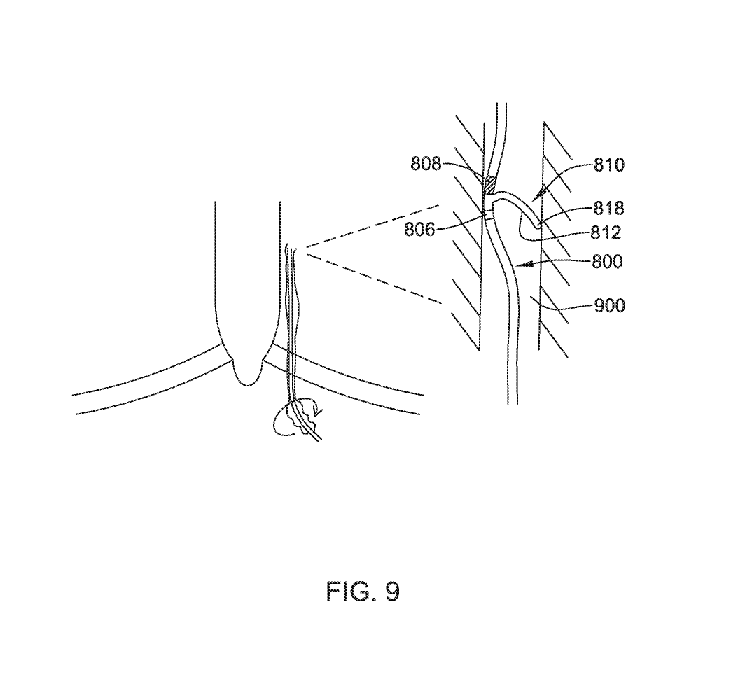

FIG. 9 shows an example of implantation of the implantable lead 800 in a blood vessel 900 (e.g., an ITV, an intercostal vein, an azygos vein, a hemiazygos vein, or an accessory hemiazygos vein). In some examples, the placement may be extravascular such as in the mediastinal space, in which case the flap 812, when deployed, may bias the lead in a desired direction such as toward the heart relative to the sternum. In this example, the flap 812 of the securing structure 810 has been deployed and is extended to bias the electrode structures 806 and 808 in a desired direction (e.g., against the right wall of the ITV). In this example, the flap 812 may self-expand on removal of an outer delivery sheath or catheter, or the flap 812 may be expanded by movement of the lead 800 relative to the sheath. Upon removal of the sheath, the flap 812 may move from a compressed, pre-deployed state to an extended, deployed state, where the distal end 818 of the flap 812 pushes against a left wall of the ITV causing the electrode structures 806 and 808 to press against the right wall of the ITV.

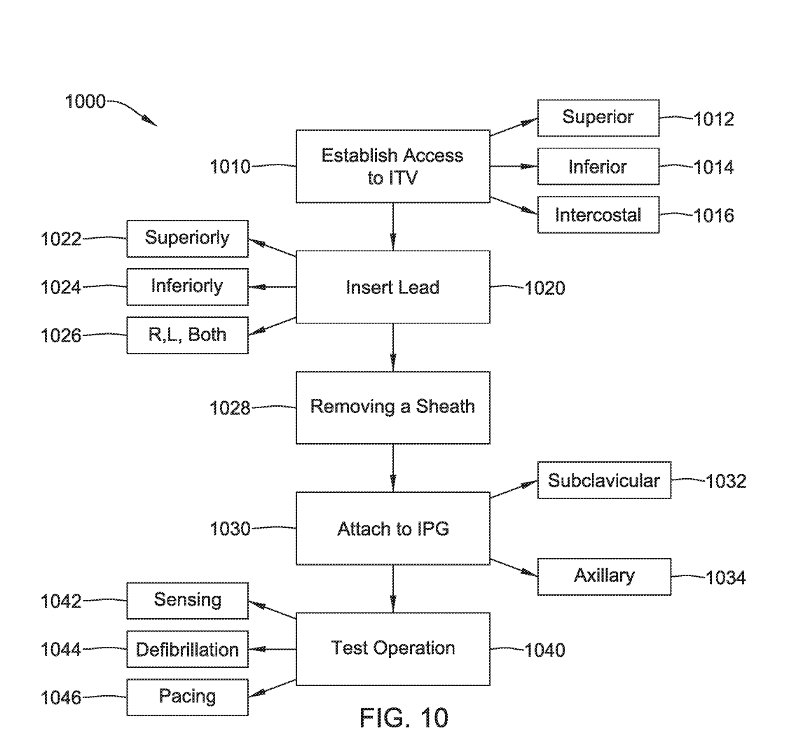

FIG. 10 is a block flow diagram for an illustrative method of implanting a lead in a patient. As shown at 1000, the method comprises establishing access to the ITV 1010, inserting a lead in the ITV 1020, removing a sheath 1028, attaching an IPG to the lead 1030, and performing test operations 1040.

For example, in some embodiments, an IRM with a securing structure (e.g., IRM 500) may have a lumen configured to receive a lead. In other embodiments, a securing structure and the lead may be formed as a single-piece or molded together. In either embodiments, a sheath may cover the securing structure before and during delivery such that the securing structure is in a compressed, pre-deployment state. Furthermore, establishing access to the ITV 1010 may include accessing from a superior position 1012 such as by entering the subclavian vein and passing through the ostium of the ITV in the brachiocephalic vein. In another example, establishing access to the ITV 1010 may include accessing from an inferior position 1014 such as by entering the superior epigastric vein or musculophrenic vein, and passing superiorly therefrom into the ITV. In some examples, access via locations 1012, and 1014 may include accessing via a second blood vessel such as by accessing superiorly 1012 by way of the subclavicular vein and brachiocephalic vein, or accessing inferiorly 1014 through the superior epigastric vein or musculophrenic vein. In still another example, establishing access to the ITV may include accessing in an intercostal space 1016 such as by penetrating an intercostal space and entering the ITV using a Seldinger technique.

In an example, inserting the lead 1020 may include insertion superiorly 1022, such as by starting in an inferior position 1012 inferior to the lower rib margin or intercostally 1016 from an inferior intercostal location, and advancing the lead in a superior direction. For another example, inserting the lead 1020 may include insertion inferiorly 1024, that is starting at a superior location 1014 or at a superior intercostal location 1016, and advancing the lead in an inferior direction. In either such example, the right ITV, left ITV, or both ITV vessels may be used, as indicated at 1026.

Other vessels and implanted lead locations may also be used (such as having a lead in the azygos, hemiazygos, or accessory hemiazygos vein, an intracardiac lead, a subcutaneous lead, and/or for placement in a coronary blood vessel). At a system level, additional devices such as a separately implanted leadless cardiac pacemaker may be included as well. In a further example, one or more of the transverse veins that flow into the ITV may be used for placement of an electrode structure or lead. For example, upon accessing an ITV, a physician may further access and emplace a lead or electrode structure into one of the intercostal veins which run along the intercostal spaces of the chest.

In an example, removing a sheath 1028 may include at least partly withdrawing the sheath to deploy the securing structure to bias the electrode structure in a desired direction. Partial removal may allow observation of whether positioning is desirable with the possibility of repositioning. If repositioning is needed the sheath may be advanced back into its original position to allow movement of the lead; once a desired position is achieved, complete removal can occur. In this example, upon removal of the sheath, the securing structure may move from the compressed, pre-deployed state to an extended, deployed state, where the securing structure pushes against a wall of the ITV causing the electrode structures to press against an opposing wall of the ITV. The sheath may be a splittable sheath if desired.

In an example, attaching to an IPG 1030 may include attaching to a canister located in a subclavicular location 1032, historically a common place to put an implanted canister for a transvenous defibrillator or pacemaker. In another example, attaching to an IPG may include attaching to a canister located in an axillary position 1034, such as that used with the S-ICD System. Other IPG locations may be used. Attachment may be directly to the IPG or to a splitter, yoke, or lead extension, if desired.

In an example, test operation 1040 may be used to verify one or both of device functionality and efficacy. For example, sensing operations 1042 may be tested and configured to check for adequate signal availability, for example, or by setting gain, filtering, or sensing vector selection parameters. For example, noise and/or signal to nois ratios may be observed to identify good cardiac signal availability for a given pair of sensing electrodes. Defibrillation operations 1044 may be tested by inducting an arrhythmia such as a ventricular fibrillation to determine whether the device will sense the arrhythmia and, if the arrhythmia is sensed, to ensure that the device can adequately provide therapy output by delivering defibrillation at a preset energy. Defibrillation testing 1044 may include determining for a given patient an appropriate defibrillation threshold, and setting a parameter for therapy delivery at some safety margin above the defibrillation threshold.

Prior transvenous systems would typically deliver up to 35 Joules of energy at most, with storage of up to 40 Joules of energy, using peak voltages in the range of up to nearly 1000 volts. The S-ICD System can deliver up to 80 Joules of energy, with 65 Joules often used for in-clinic system testing, with a peak voltage in the range of 1500 volts. The ITV location may facilitate energy levels similar to those of traditional transvenous systems (5-35 Joules, approximately), or may be somewhat higher (5 to about 50 joules, for example), or may still be higher (10 to about 60 joules, for example). Pacing thresholds may also be closer to those for traditional transvenous systems than the more recent S-ICD System.

In an example, pacing testing operation 1046 may include determining which, if any, available pacing vectors are effective to provide pacing capture. If desired, parameters may be tested as well to determine and optimize settings for delivery of cardiac resynchronization therapy. This may include testing of pacing thresholds to optimize energy usage and delivery, as well as checking that adverse secondary effects, such as patient sensation of the delivered pacing or inadvertent stimulation of the phrenic nerve, diaphragm or skeletal muscles are avoided.

Some embodiments of the present invention may take the form of an implantation tool set configured for use in implanting a cardiac device, such as a lead, into an ITV. Some such embodiments may include an introducer sheath. Some such embodiments may include a guide catheter. Some such embodiments may include a guidewire. Some such embodiments may further include a tool set for performing a Seldinger technique to access a blood vessel percutaneously.

Some embodiments of the present invention take the form of an implantable cardiac stimulus device comprising a lead and an implantable canister for coupling to the lead, the implantable canister housing operational circuitry configured to deliver output therapy in the form of at least one of bradycardia pacing, anti-tachycardia pacing, cardiac resynchronization therapy, or defibrillation, using a lead implanted in an ITV and a canister implanted in a patient.

As used herein, a coil electrode may be a helically wound element, filament, or strand. The filament forming the coil may have a generally round or a generally flat (e.g. rectangular) cross-sectional shape, as desired. However, other cross-sectional shapes may be used. The coil electrode may have a closed pitch, or in other words, adjacent windings may contact one another. Alternatively, the coil electrode may have an open pitch such that adjacent windings are spaced a distance from one another. The pitch may be uniform or varied along a length of the coil electrode. A varied pitch may be gradual tapered changes in pitch or abrupt or step-wise changes in pitch.

A coil electrode may have a length L that is generally larger than a width W. Round, oval or flattened coil electrodes may be used. Coil electrodes may have a length in the range of one to ten centimeters. In an example, a coil having a six or eight centimeter length may be used. In another example, a lead may have two four centimeter coils. Coils and leads may be in the range of four to ten French, or larger or smaller, in outer profile.

Coils and leads may be coated. For example, a thin permeable membrane may be positioned over a shock coil or other electrode and/or other portions of the lead to inhibit or to promote tissue ingrowth. Coatings, such as, but not limited to expanded polytetrafluoroethylene (ePTFE) may also be applied to the coil and/or lead to facilitate extraction and/or to reduce tissue ingrowth. In some embodiments, one or more of the electrodes, whether coils, rings, or segmented electrodes, include a high capacitive coating such as, but not limited to iridium oxide (IrOx), titanium nitride (TiN), or other “fractal” coatings which may be used, for example, to improve electrical performance. Steroidal and antimicrobial coatings may be provided as well.

The various components of the devices/systems disclosed herein may include a metal, metal alloy, polymer, a metal-polymer composite, ceramics, combinations thereof, and the like, or other suitable material. Some examples of suitable metals and metal alloys include stainless steel, such as 304V, 304L, and 316LV stainless steel; mild steel; nickel-titanium alloy such as linear-elastic and/or super-elastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R30035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003 such as ELGILOY®, PHYNOX®, and the like); platinum enriched stainless steel; titanium; combinations thereof; and the like; or any other suitable material.

Some examples of suitable polymers for use in the leads discussed above may include polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane (for example, Polyurethane 85A), polypropylene (PP), polyvinylchloride (PVC), polyether-ester (for example, ARNITEL® available from DSM Engineering Plastics), ether or ester based copolymers (for example, butylene/poly(alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), Marlex high-density polyethylene, Marlex low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon), perfluoro(propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS A), polycarbonates, ionomers, biocompatible polymers, other suitable materials, or mixtures, combinations, copolymers thereof, polymer/metal composites, and the like.

In at least some embodiments, portions or all of the accessory devices and their related components may be doped with, made of, or otherwise include a radiopaque material. Radiopaque materials are understood to be materials capable of producing a relatively bright image on a fluoroscopy screen or another imaging technique during a medical procedure. This relatively bright image aids the user of the accessory devices and their related components in determining its location. Some examples of radiopaque materials can include, but are not limited to, gold, platinum, palladium, tantalum, tungsten alloy, polymer material loaded with a radiopaque filler, and the like. Additionally, other radiopaque marker bands and/or coils may also be incorporated into the design of the accessory devices and their related components to achieve the same result.

Any guidewire, introducer sheath, and/or guide catheter design suitable for medical interventions may be used for accessing the venous structures discussed herein.

The implantable systems shown above may include an implantable pulse generator (IPG) adapted for use in a cardiac therapy system. The IPG may include a hermetically sealed canister that houses the operational circuitry of the system. The operational circuitry may include various components such as a battery, and one or more of low-power and high-power circuitry. Low-power circuitry may be used for sensing cardiac signals including filtering, amplifying and digitizing sensed data. Low-power circuitry may also be used for certain cardiac therapy outputs such as pacing output, as well as an annunciator, such as a beeper or buzzer, telemetry circuitry for RF, conducted or inductive communication (or, alternatively, infrared, sonic and/or cellular) for use with a non-implanted programmer or communicator. The operational circuitry may also comprise memory and logic circuitry that will typically couple with one another via a control module which may include a controller or processor. High power circuitry such as high power capacitors, a charger, and an output circuit such as an H-bridge having high power switches may also be provided for delivering, for example, defibrillation therapy. Other circuitry and actuators may be included such as an accelerometer or thermistor to detected changes in patient position or temperature for various purposes, output actuators for delivering a therapeutic substance such as a drug, insulin or insulin replacement, for example.

Some illustrative examples for hardware, leads and the like for implantable defibrillators may be found in commercially available systems such as the Boston Scientific Teligen™ ICD and Emblem S-ICD™ System, Medtronic Concerto™ and Virtuoso™ systems, and St. Jude Medical Promote™ RF and Current™ RF systems, as well as the leads provided for use with such systems.

Each of these non-limiting examples can stand on its own, or can be combined in various permutations or combinations with one or more of the other examples.

The above detailed description includes references to the accompanying drawings, which form a part of the detailed description. The drawings show, by way of illustration, specific embodiments in which the invention can be practiced. These embodiments are also referred to herein as “examples.” Such examples can include components in addition to those shown or described. However, the present inventors also contemplate examples in which only those components shown or described are provided. Moreover, the present inventors also contemplate examples using any combination or permutation of those components shown or described (or one or more aspects thereof), either with respect to a particular example (or one or more aspects thereof), or with respect to other examples (or one or more aspects thereof) shown or described herein.

In the event of inconsistent usages between this document and any documents so incorporated by reference, the usage in this document controls. In this document, the terms “a” or “an” are used, as is common in patent documents, to include one or more than one, independent of any other instances or usages of “at least one” or “one or more.” Moreover, in the following claims, the terms “first,” “second,” and “third,” etc. are used merely as labels, and are not intended to impose numerical requirements on their objects.

Method examples described herein can be machine or computer-implemented at least in part. Some examples can include a computer-readable medium or machine-readable medium encoded with instructions operable to configure an electronic device to perform methods as described in the above examples. An implementation of such methods can include code, such as microcode, assembly language code, a higher-level language code, or the like. Such code can include computer readable instructions for performing various methods. The code may form portions of computer program products. Further, in an example, the code can be tangibly stored on one or more volatile, non-transitory, or non-volatile tangible computer-readable media, such as during execution or at other times. Examples of these tangible computer-readable media can include, but are not limited to, hard disks, removable magnetic or optical disks, magnetic cassettes, memory cards or sticks, random access memories (RAMs), read only memories (ROMs), and the like.

The above description is intended to be illustrative, and not restrictive. For example, the above-described examples (or one or more aspects thereof) may be used in combination with each other. Other embodiments can be used, such as by one of ordinary skill in the art upon reviewing the above description. The Abstract is provided to comply with 37 C.F.R. § 1.72(b), to allow the reader to quickly ascertain the nature of the technical disclosure. It is submitted with the understanding that it will not be used to interpret or limit the scope or meaning of the claims.

Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description as examples or embodiments, with each claim standing on its own as a separate embodiment, and it is contemplated that such embodiments can be combined with each other in various combinations or permutations. The scope of the invention should be determined with reference to the appended claims, along with the full scope of equivalents to which such claims are entitled.

The claimed invention is:

1. A channel body for use in an implantable medical device comprising:
a body having an outer surface and a longitudinal axis extending between a proximal end and a distal end; and
a plurality of channels extending from the proximal end to the distal end of the body and radially spaced from one another, each of the channels being configured to receive an electrode structure therein such that the body electrically isolates the electrode structures from one another and enables contact between tissue of a patient and the electrode structures in the channels.

2. A channel body as in claim 1, wherein the body further comprises a lumen extending from the proximal end to the distal end along the longitudinal axis and configured to receive a positioning mechanism.

3. A lead assembly for use in an implantable medical device system comprising:
the channel body as in claim 1;
a lead body extending from a proximal end to a distal end, the channel body secured at or near the distal end of the lead body; and
a plurality of conductors extending through the lead body to the channel body which are coupled to the electrode structures.

4. The lead assembly of claim 3 wherein the lead assembly has a first length, and the channel body has a second length, and the second length is less than half of the first length.

5. The lead assembly of claim 3 further comprising an outer sheath having one or more portions made up of a conductive material, and one or more portions made up of a dielectric material.

6. The lead assembly of claim 3 wherein the channels of the channel body extend in a longitudinal direction along the channel body.

7. The lead assembly of claim 3 wherein the channels of the channel body extend helically about the channel body.

8. The lead assembly of claim 3 comprising a connector at a proximal end thereof for coupling to an implantable medical device, the connector having contacts corresponding to one or more of the plurality of conductors.

9. An implantable medical device system comprising:
an implantable pulse generator housing operational circuitry for the implantable medical device system; and
a lead assembly as in claim 8, wherein:
the implantable pulse generator comprises a port for receiving the connector of the lead assembly, the connector and port configured to electrically couple the plurality of conductors to the operational circuitry; and
the operational circuitry is configured to deliver therapy using the electrode structures.

10. The implantable medical device system of claim 9 wherein the implantable pulse generator is configured to deliver a pacing therapy by selecting a first electrode structure on the channel body as a first output pole, and selecting a second electrode structure on the channel body as a second output pole.

11. The implantable medical device system of claim 10 wherein the operational circuitry is configured to measure impedances between at least first and second selected ones of the electrode structures on the channel body and determine from the measured impedances which of the electrode structures to use in pacing therapy delivery.

12. The implantable medical device system of claim 9 wherein the implantable pulse generator is configured to deliver a defibrillation therapy by linking at least two of the plurality of electrode structures on the channel body in common, and using an electrode on the implantable pulse generator as an opposing electrode thereto.

13. An implantable medical device system comprising:
an implantable pulse generator housing operational circuitry for the implantable medical device system; and
a lead assembly as in claim 8, wherein:
the implantable pulse generator comprises a port for receiving the connector of the lead assembly, the connector and port configured to electrically couple the plurality of conductors to the operational circuitry; and
the operational circuitry is configured to sense cardiac activity using selected ones of the electrode structures.

14. The channel body of claim 1 wherein the channels of the channel body extend in a longitudinal direction along the channel body.

15. The channel body of claim 1 wherein the channels of the channel body extend helically about the channel body.

16. A method of delivering therapy to a patient using an implantable cardiac stimulus system comprising an implantable pulse generator and a lead assembly, the lead assembly comprising a lead body extending from a proximal end adapted for attachment to the pulse generator to a distal portion having a channel body with a plurality of channels on a surface thereof having a plurality of electrode structures in the plurality of channels coupled to a plurality of conductors that extend from the proximal end of the lead to the channel body, the method comprising:
selecting at least one of the plurality of electrode structures as a first output pole for delivery of therapy;
selecting a second electrode or electrodes as a second output pole for delivery of therapy; and
delivering therapy between the first and second output poles.

17. The method of claim 16 wherein:
the therapy is a pacing therapy;
the first output pole uses a selected first one of the plurality of electrode structures; and
the second output pole uses a selected second one of the plurality of electrode structures.

18. The method of claim 16 wherein:
the therapy is a defibrillation therapy;
the pulse generator comprises a housing having an electrode usable for delivery of therapy;
the first output pole uses at least two of the plurality of electrode structures in common; and
the second output pole uses the housing of the pulse generator.

19. A method of sensing cardiac activity of a patient using an implantable cardiac stimulus system comprising an implantable pulse generator and a lead assembly, the lead assembly comprising a lead body extending from a proximal end adapted for attachment to the pulse generator to a distal portion having a channel body with a plurality of channels on a surface thereof having a plurality of electrode structures in the plurality of channels coupled to a plurality of conductors that extend from the proximal end of the lead to the channel body, the method comprising:
selecting a first one of the plurality of electrode structures as a first sensing electrode;
selecting a second one of the plurality of electrode structures as a second sensing electrode; and
sensing an electrical signal between the first and second sensing electrodes.

20. The method of claim 19 wherein the lead assembly distal portion is disposed subcutaneously without entering or contacting the heart of the patient.
